(19) United States
(12) Patent Application Publication
(10) Pub. No.: US 2006/0142647 A1
(43) Pub. Date: Jun. 29, 2006

(54) Diagnosis aiding apparatus, method, and computer program

(75) Inventor: Akira Oosawa, Kanagawa-ken (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/317,025

(22) Filed: Dec. 27, 2005

(30) Foreign Application Priority Data
Dec. 27, 2004 (JP) 376605/2004

(51) Int. Cl. A61B 5/00

(52) U.S. Cl. 600/300; 128/920

(57) ABSTRACT

Multiple pieces of diagnostic information, which are necessary for disease diagnoses, and at least one piece of disease information, which represents a disease to be diagnosed in accordance with the diagnostic information, are stored. Link information is stored for each piece of disease information. The link information links the multiple pieces of the diagnostic information together in accordance with relationships among the pieces of the diagnostic information to be utilized at the time of a diagnosis of each disease and/or links the diagnostic information and the disease information together in accordance with relationships between the diagnostic information and the disease information. A probability of occurrence of a disease in accordance with the link information is stored previously. The probability of occurrence of each disease associated with inputted diagnostic information is acquired in accordance with the link information.

### BACKGROUND OF THE INVENTION

1. Field of the Invention

This invention relates to a diagnosis aiding apparatus, method, and computer program for aiding medical doctors to make diagnoses of diseases of patients.

2. Description of the Related Art

In the field of medical diagnoses, in cases where judgments as to names of diseases are to be made in accordance with conditions of diseases of patients, results of pathological examinations, and the like, the judgments have heretofore been made in accordance with chief complaints made by the patients, such as complaints of feeling of nausea and complaints of feeling heavy in the heads, and results of diagnoses of the patients made by the medical doctors. Also, when necessary, results of image diagnoses made on radiation images of the patients, and the like, have heretofore been taken into consideration for specifying the names of the corresponding diseases.

However, in cases where judgments are synthetically made in accordance with the available results, since the obtained results do not necessarily correspond to only one name of the disease and often correspond to a plurality of diseases, and since symptoms may be approximately identical for different diseases, it may often occur that the names of diseases are not capable of being specified. Also, in cases where diseases with approximately identical symptoms are included in the diseases corresponding to the obtained results, the number of the names of suspected diseases becomes large even further.

Recently, as computed radiography (CR) apparatuses, computed tomography (CT) scanners, and magnetic resonance imaging (MRI) apparatuses have become popular, rapid advances have been made in digitization of medical images, and large numbers of medical images ranging from simple recording photographs to angiograms have been digitized and stored in the form of digital image signals. In order for the digitized medical images to be utilized efficiently in making diagnoses, processing of image information has been standardized, e.g., as the digital imaging and communications in medicine (DICOM) standards, such that image transmission and accumulation are capable of being performed quickly, and a picture archiving and communication system (PACS), which enables mutual connections among various computer systems, has been developed.

Also, in parallel with the digitization of the medical images, rapid advances have been made in introduction of medical systems into medical institutions. Hospital information systems (HIS) for performing various operations ranging from medical fee processing to business management are thus becoming popular. Further, radiology information systems (RIS) for furnishing diagnosis aiding environments, which enable advanced information supply within hospitals and within departments, are becoming popular. Furthermore, conventional clinical charts have been replaced by digitized electronic clinical charts, and information representing clinical histories, medical prescription histories, and the like, have been digitized and stored as digital data.

As the information necessary for diagnoses of diseases are digitized and stored as digital data as described above, there have been proposed the techniques for storing the information, which represents past opinions, and the information, which represents details of definite diagnoses, and specifying the diseases through comparison with past disease cases.

One of the techniques for storing the information, which represents past opinions, and the information, which represents details of definite diagnoses, and specifying the diseases through comparison with past disease cases is proposed in, for example, Japanese Unexamined Patent Publication No. 6(1994)-292656. With the proposed technique, firstly, opinions and corresponding definite diagnoses (e.g., diagnoses as being cerebral infarction) with respect to past disease cases are symbolized and stored in data base. For example, the symbolization is set such that an image recording apparatus (e.g., an X-ray image recording apparatus) is represented as classification into large groups, such that a site (e.g., a brain) is represented as classification into medium groups, such that a disease name (e.g., a subarachnoid space) is represented as classification into small groups, such that a detailed opinion (e.g., local stricture of . . . ) is represented as classification into fine groups, and such that a detailed classification of a disease name (e.g., right Sylvius fissure) is represented as sub-classification. In this manner, all of the past disease cases are symbolized and stored in the data base. In cases where a diagnosis is to be made for a new patient, an image diagnosis is performed on the image of the new patient, and the corresponding classification symbols are inputted. The corresponding past disease cases are thus retrieved from the data base, and all of the disease names in all of the disease cases associated with the identical opinion having been made in the past are investigated. In this manner, a probability of occurrence of each disease (i.e., the opinion probability) is calculated. Also, with respect to each of the disease names having been retrieved, the probability of occurrence of each disease in the facility (i.e., the prior probability) is calculated. Further, a disease probability with respect to the new patient image is calculated from the opinion probability and the prior probability, and disease names are displayed in the order of decreasing probability.

Also, a different technique for storing the information, which represents past opinions, and the information, which represents details of definite diagnoses, and specifying the diseases through comparison with past disease cases is proposed in, for example, Japanese Unexamined Patent Publication No. 2002-032476. With the proposed technique, with respect to the past diseases, diagnostic opinions (#+small letters and numerals for disease names), image diagnosis results ($+small letters and numerals for disease names), medical interview results (%+small letters and numerals for disease names), pathological examination results (&+small letters and numerals for disease names), and other kinds of information (@+small letters and numerals for disease names) are stored in a data base. Also, it is made clear which typical disease condition information and which image data correspond to which disease name. In cases where a diagnosis is to be made on a new patient, the medical doctor inputs diagnostic opinions, image diagnosis results, medical interview results, pathological examination results, and other kinds of information and performs retrieval from the data base. In this manner, the disease names and the image data corresponding to the inputted information are listed and displayed.

However, with each of the aforesaid two techniques for storing the information, which represents past opinions, and the information, which represents details of definite diagnoses, and specifying the diseases through comparison with past disease cases, the past disease cases are retrieved from the data base by the utilization of the retrieval items given by the medical doctor. Therefore, an opinion, which has failed to be inputted, is not capable of being utilized as the information for specifying the disease. For example, in cases where an opinion with respect to a radiography and an opinion with respect to a CT image have already been stored in the data base as the opinions for a certain patient, if only the opinion with respect to the radiography is inputted, the opinion with respect to the CT image will not be capable of being utilized for the retrieval unless the medical doctor inputs the opinion with respect to the CT image. In ordinary diagnoses, the accuracy with which a disease is specified is capable of being enhanced by the utilization of both the radiography and the CT image. Similarly, in cases where past disease cases are to be retrieved from the data base, the accuracy with which a disease is specified is expected to be enhanced by the utilization of both the radiography and the CT image for the specifying of the disease.

Recently, systems in which the HIS, the RIS, the modalities, the PACS, and the like, are connected with one another are being constructed. Also, various information systems, which have heretofore been located at different buildings, different areas, and different business sections within a hospital, are nowadays connected with one another through a network. Therefore, a plurality of pieces of information concerning an identical patient, which have heretofore been stored at various different locations within the hospital, are capable of being collected at one place through the network. Further, in cases where diagnosis aiding functions with a computer are to be utilized, a considerable time and labor will be required for the medical doctor to input all of the retrieval items. Accordingly, it is desired that, in cases where certain retrieval items are inputted, the other retrieval items necessary for a judgment are capable of being made up by a computer, and various pieces of information are capable of being actively collected for diagnoses.

Furthermore, in cases where a disease is not capable of being specified with synthetic diagnosis made from chief complaints of a patient and available examination results, other additional pieces of information have heretofore been acquired from additional examinations, a past clinical history, and the like, and utilized for the diagnosis. However, it may often occur that a medical doctor having little experience is not capable of accurately making a judgment as to which additional examinations are to be performed. Such that the medical doctor is capable of making an accurate judgment as to which additional examinations are to be performed, it has heretofore been necessary for the medical doctor to enrich the experience.

### SUMMARY OF THE INVENTION

The primary object of the present invention is to provide a diagnosis aiding apparatus, wherein diagnoses are aided such that the diagnoses of diseases are capable of being made accurately by use of information concerning diseases, which information has been stored in a computer.

Another object of the present invention is to provide a diagnosis aiding method, wherein diagnoses are aided such that the diagnoses of diseases are capable of being made accurately by use of information concerning diseases, which information has been stored in a computer.

A further object of the present invention is to provide a computer program for causing a computer to execute the diagnosis aiding method.

The present invention provides a diagnosis aiding apparatus, comprising:

i) link information storing means for storing a plurality of pieces of diagnostic information, which are necessary for disease diagnoses, and at least one piece of disease information, which represents a disease to be diagnosed in accordance with the diagnostic information, and storing link information with respect to each piece of the disease information,

the link information linking the plurality of pieces of the diagnostic information with one another in accordance with relationships among the plurality of pieces of the diagnostic information, which are to be utilized at the time of a diagnosis of each disease, and/or linking the plurality of pieces of the diagnostic information and the disease information with each other in accordance with relationships between the plurality of pieces of the diagnostic information and the disease information,

ii) probability storing means for previously storing a probability of occurrence, with which the disease occurs in accordance with the plurality of pieces of the diagnostic information, and a probability of occurrence, with which the disease occurs in accordance with a combination of the plurality of pieces of the diagnostic information corresponding to the link information,

iii) input means for inputting at least one piece of the diagnostic information among the plurality of pieces of the diagnostic information, and

iv) probability acquiring means for retrieving the probability of occurrence, which is associated with the diagnostic information having been inputted from the input means, from the probability storing means, and acquiring the probability of occurrence with respect to each disease by use of the thus retrieved probability of occurrence and in accordance with the link information of each piece of the disease information.

The present invention also provides a diagnosis aiding method, comprising the steps of:

i) storing a plurality of pieces of diagnostic information, which are necessary for disease diagnoses, and at least one piece of disease information, which represents a disease to be diagnosed in accordance with the diagnostic information,

ii) storing link information with respect to each piece of the disease information,

the link information linking the plurality of pieces of the diagnostic information with one another in accordance with relationships among the plurality of pieces of the diagnostic information, which are to be utilized at the time of a diagnosis of each disease, and/or linking the plurality of pieces of the diagnostic information and the disease information with each other in accordance with relationships between the plurality of pieces of the diagnostic information and the disease information,

iii) previously storing a probability of occurrence, with which the disease occurs in accordance with the plurality of pieces of the diagnostic information, and a probability of occurrence, with which the disease occurs in accordance with a combination of the plurality of pieces of the diagnostic information corresponding to the link information,

iv) inputting at least one piece of the diagnostic information among the plurality of pieces of the diagnostic information,

v) retrieving the probability of occurrence, which is associated with the diagnostic information having been inputted from the input means, from the probability storing means, and

vi) acquiring the probability of occurrence with respect to each disease by use of the thus retrieved probability of occurrence and in accordance with the link information of each piece of the disease information.

The present invention further provides a computer program for operating a computer provided with:

i) link information storing means for storing a plurality of pieces of diagnostic information, which are necessary for disease diagnoses, and at least one piece of disease information, which represents a disease to be diagnosed in accordance with the diagnostic information, and storing link information with respect to each piece of the disease information,

the link information linking the plurality of pieces of the diagnostic information with one another in accordance with relationships among the plurality of pieces of the diagnostic information, which are to be utilized at the time of a diagnosis of each disease, and/or linking the plurality of pieces of the diagnostic information and the disease information with each other in accordance with relationships between the plurality of pieces of the diagnostic information and the disease information, and

ii) probability storing means for previously storing a probability of occurrence, with which the disease occurs in accordance with the plurality of pieces of the diagnostic information, and a probability of occurrence, with which the disease occurs in accordance with a combination of the plurality of pieces of the diagnostic information corresponding to the link information,

wherein the computer program causes the computer to function as:

a) input means for inputting at least one piece of the diagnostic information among the plurality of pieces of the diagnostic information, and

b) probability acquiring means for retrieving the probability of occurrence, which is associated with the diagnostic information having been inputted from the input means, from the probability storing means, and acquiring the probability of occurrence with respect to each disease by use of the thus retrieved probability of occurrence and in accordance with the link information of each piece of the disease information.

The term “diagnostic information” as used herein means the information to be used for making diagnoses of various diseases. Examples of the diagnostic information include data on opinions of medical doctors having diagnosed patients, which data have been encoded and inputted from input devices, data on inputted values representing examination results, and data on processing results obtained with computer aided design (CAD).

The term “acquiring a probability of occurrence with respect to each disease by use of a retrieved probability of occurrence and in accordance with link information of each piece of disease information” as used herein means that the probability of occurrence of the disease represented by each piece of disease information, with which the inputted diagnostic information is linked, is acquired. For example, in cases where the diagnostic information is linked with the link information of both the lung cancer and the tuberculosis, both the probability of occurrence of the lung cancer and the probability of occurrence of the tuberculosis maybe acquired. Alternatively, only either one of the probability of occurrence of the lung cancer and the probability of occurrence of the tuberculosis may be acquired.

The diagnosis aiding apparatus in accordance with the present invention may be modified such that the apparatus further comprises displaying means for displaying the disease information in the order of decreasing probability of occurrence of each disease, which probability of occurrence has been acquired by the probability acquiring means.

Also, the diagnosis aiding apparatus in accordance with the present invention may be modified such that the apparatus further comprises selection means for selecting a certain piece of the diagnostic information, which is other than the at least one piece of the diagnostic information having been inputted from the input means, from among the plurality of pieces of the diagnostic information, which are linked by the link information and are to be used at the time of the diagnosis of a predetermined disease,

the certain piece of the diagnostic information being of the kind such that, in cases where the certain piece of the diagnostic information is added to the at least one piece of the diagnostic information having been inputted from the input means and is utilized for the acquisition of the probability of occurrence of the predetermined disease made by the probability acquiring means, the probability of occurrence of the predetermined disease, which probability is higher than the probability of occurrence of the predetermined disease having been acquired from the probability acquiring means by use of the at least one piece of the diagnostic information having been inputted from the input means, is acquired from the probability acquiring means.

With each of the diagnosis aiding apparatus, method, and computer program in accordance with the present invention, the link information, which links the plurality of pieces of the diagnostic information and the disease information with each other in accordance with relationships between the plurality of pieces of the diagnostic information necessary for disease diagnoses and the diseases, is stored. Also, the probability of occurrence, with which each disease occurs in accordance with the combination of the plurality of pieces of the diagnostic information corresponding to the link information, is acquired. Therefore, the possibility of being the disease is capable of being judged from the inputted piece of the diagnostic information. Also, the probability of occurrence of every disease, with which the inputted piece of the diagnostic information is linked, is capable of being acquired. Specifically, besides the probability of occurrence of only one disease, the probabilities of occurrence of a plurality of diseases are capable of being acquired. Accordingly, attention is capable of being called to the possibility of diseases other than the disease having been expected by the medical doctor.

In cases where the disease information is displayed in the order of decreasing probability of occurrence of each disease, it becomes possible to make a judgment as to which disease has the highest possibility.

Also, the certain piece of the diagnostic information, which is other than the at least one piece of the diagnostic information having been inputted from the input means, may be selected from among the plurality of pieces of the diagnostic information, which are to be used at the time of the diagnosis of the predetermined disease. The certain piece of the diagnostic information is of the kind such that, in cases where the certain piece of the diagnostic information is added to the at least one piece of the diagnostic information having been inputted and is utilized for the acquisition of the probability of occurrence of the predetermined disease, the probability of occurrence of the predetermined disease is acquired, which probability is higher than the probability of occurrence of the predetermined disease having been acquired by use of the at least one piece of the diagnostic information having been inputted. In such cases, the certain piece of the diagnostic information among the plurality of pieces of the diagnostic information necessary for the disease diagnoses, which certain piece of the diagnostic information has not been inputted, is capable of being extracted, and a diagnostic item having a high certainty of being necessary is capable of being instructed to the medical doctor and other persons.

### BRIEF DESCRIPTION OF THE DRAWINGS

FIG. 1 is a block diagram showing an embodiment of the diagnosis aiding apparatus in accordance with the present invention, which is connected to information systems,

FIG. 2 is a block diagram showing the embodiment of the diagnosis aiding apparatus in accordance with the present invention,

FIG. 3 is an explanatory view showing an example of how diagnostic information is symbolized,

FIGS. 4A, 4B, and 4C are explanatory views showing an example of how position information is automatically acquired from a chest image by use of functions of a CAD apparatus,

FIG. 5 is an explanatory view showing an example of link information, which links diagnostic information and disease information together,

FIG. 6A is an explanatory view showing examples of pieces of diagnostic information,

FIG. 6B is an explanatory view showing an example of the displaying of probabilities of occurrence of diseases, and

FIG. 7 is an explanatory view showing an example of the displaying of an instruction for requesting the inputting of diagnostic information.

### DESCRIPTION OF THE PREFERRED EMBODIMENTS

The present invention will hereinbelow be described in further detail with reference to the accompanying drawings.

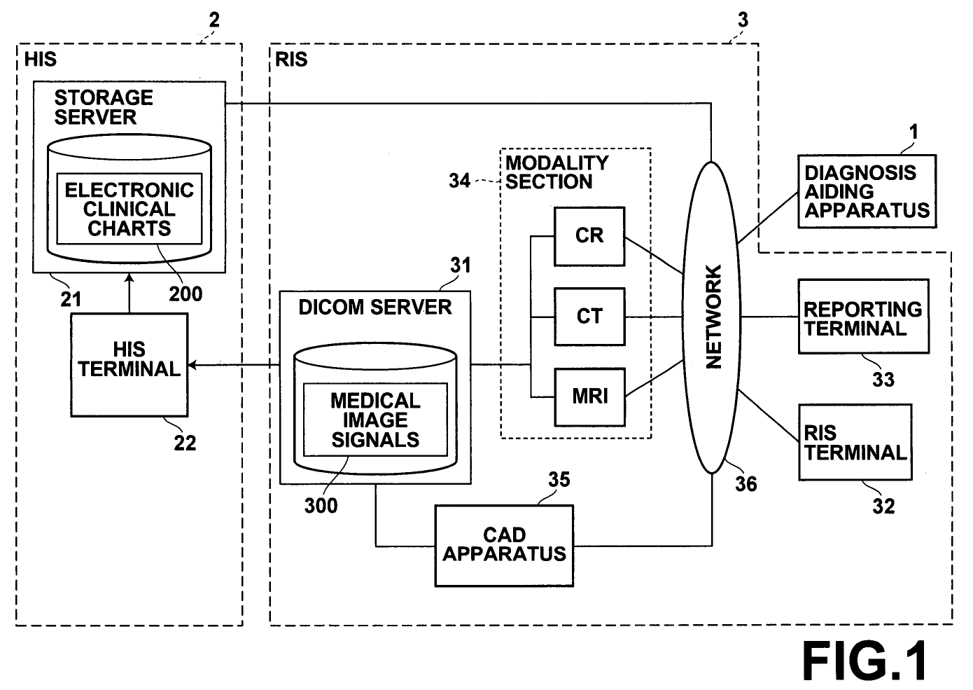

FIG. 1 is a block diagram showing an embodiment of the diagnosis aiding apparatus in accordance with the present invention, which is connected to information systems. With reference to FIG. 1, a diagnosis aiding apparatus 1, which is an embodiment of the diagnosis aiding apparatus in accordance with the present invention, is connected to information systems, such as an HIS 2 and an RIS 3, through a network. In the information systems, an HIS terminal 22, an RIS terminal 32, and a reporting terminal 33 are located at different positions in accordance with buildings, areas, and business stations in each hospital. The HIS terminal 22, the RIS terminal 32, and the reporting terminal 33 are connected with one another through a network 36. Also, the RIS 3 is provided with a modality section 34, which comprises various modalities, such as a CR apparatus, a CT scanner, and an MRI apparatus, for recording the images of patients. The RIS 3 is also provided with a DICOM server 31 for storing medical image signals 300, which represent medical images of the patients. Further, the HIS 2 is provided with a storage server 21 for storing electronic clinical charts 200.

By way of example, in cases where acceptance information is inputted from the HIS terminal 22, the acceptance information is registered in the electronic clinical charts 200 of the storage server 21. Also, information, which represents the details of diagnoses having been made at each of consultation departments, and information, which represents the results of medical examinations, are recorded as the electronic clinical charts 200. Further, order information is inputted from the RIS terminal 32, and the RIS terminal 32 gives instructions for the image recording in accordance with the order information to the modality section 34. The medical image signals 300 representing the medical images having been recorded by use of the modalities of the modality section 34 are sent from the modalities of the modality section 34 into the DICOM server 31 and stored in the DICOM server 31.

The RIS terminal 32 has the functions for fetching the medical image signals 300, which are necessary for the examinations and the diagnoses, from the DICOM server 31 and displaying visible medical images represented by the medical image signals 300. The reporting terminal 33 has the functions for displaying the states of the examinations and the consultations. In cases where instructions are given from the RIS terminal 32 to a CAD apparatus 35 when necessary, various kinds of image processing are capable of being performed on the medical images represented by the medical image signals 300 having been stored in the DICOM server 31. The results of the image processing are capable of being found at the RIS terminal 32 and the reporting terminal 33. Also, the information representing the examination results and the information concerning the states of execution of examinations are registered as the electronic clinical charts 200.

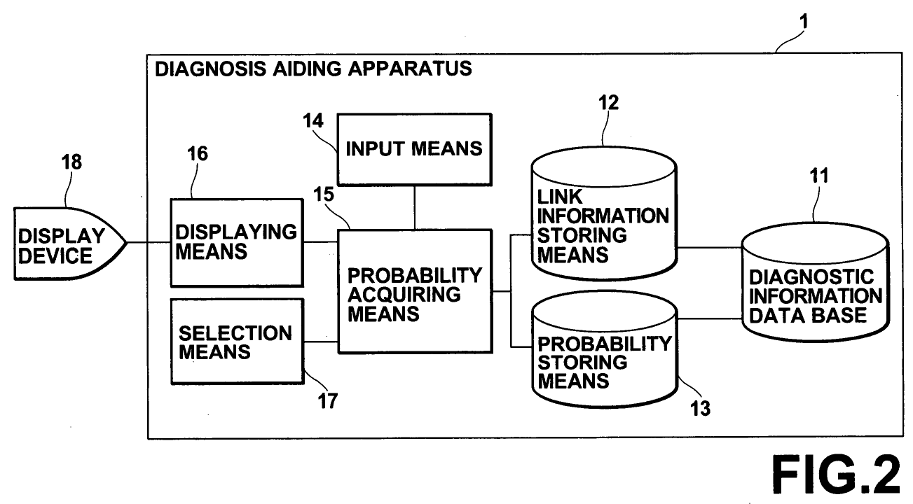

FIG. 2 is a block diagram showing the embodiment of the diagnosis aiding apparatus in accordance with the present invention. As illustrated in FIG. 2, the diagnosis aiding apparatus 1 comprises a diagnostic information data base 11, which acts as diagnostic information storing means. The diagnostic information database 11 stores a plurality of pieces of diagnostic information, which are necessary for diagnoses and represent diagnostic opinions, results of medical interviews, and the like, having been obtained for disease diagnoses, in accordance with clinical charts concerning diagnoses having been made in the past, and the like. The diagnosis aiding apparatus 1 also comprises link information storing means 12. The link information storing means 12 stores at least one piece of disease information, which represents a disease to be diagnosed in accordance with the past diagnostic information having been stored in the diagnostic information data base 11. The link information storing means 12 also stores link information with respect to each piece of the disease information. The link information links the plurality of pieces of the diagnostic information with one another in accordance with relationships among the plurality of pieces of the diagnostic information, which are to be utilized at the time of a diagnosis of each disease. The link information also links the plurality of pieces of the diagnostic information and the disease information with each other in accordance with relationships between the plurality of pieces of the diagnostic information and the disease information.

The diagnosis aiding apparatus 1 further comprises probability storing means 13 for previously storing a probability of occurrence, with which the disease occurs in accordance with the plurality of pieces of the diagnostic information, and a probability of occurrence, with which the disease occurs in accordance with a combination of the plurality of pieces of the diagnostic information corresponding to the link information. The diagnosis aiding apparatus 1 still further comprises input means 14 for inputting at least one piece of the diagnostic information among the plurality of pieces of the diagnostic information. The diagnosis aiding apparatus 1 also comprises probability acquiring means 15. The probability acquiring means 15 retrieves the probability of occurrence, which is associated with the diagnostic information having been inputted from the input means 14, from the probability storing means 13. The probability acquiring means 15 also acquires the probability of occurrence with respect to each disease by use of the thus retrieved probability of occurrence and in accordance with the link information of each piece of the disease information.

The diagnosis aiding apparatus 1 further comprises displaying means 16 for displaying the disease information in the order of decreasing probability of occurrence of each disease, which probability of occurrence has been acquired by the probability acquiring means 15. The disease information is thus displayed on a display device 18, or the like. The diagnosis aiding apparatus 1 still further comprises selection means 17 for selecting a certain piece of the diagnostic information, which is other than the at least one piece of the diagnostic information having been inputted from the input means 14, from among the plurality of pieces of the diagnostic information, which are linked by the link information and are to be used at the time of the diagnosis of a predetermined disease. The certain piece of the diagnostic information, which is selected by the selection means 17, is of the kind such that, in cases where the certain piece of the diagnostic information is added to the at least one piece of the diagnostic information having been inputted from the input means 14 and is utilized for the acquisition of the probability of occurrence of the predetermined disease made by the probability acquiring means 15, the probability of occurrence of the predetermined disease, which probability is higher than the probability of occurrence of the predetermined disease having been acquired from the probability acquiring means 15 by use of the at least one piece of the diagnostic information having been inputted from the input means 14, is acquired from the probability acquiring means 15.

1) Preparation of Diagnostic Information Data Base

Firstly, how the diagnostic information data base 11 is prepared will be described hereinbelow.

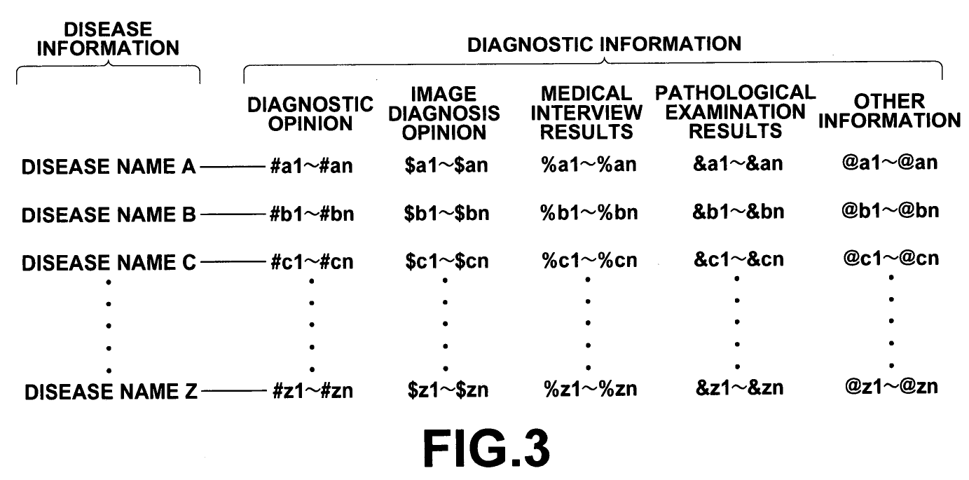

Specifically, the plurality of pieces of the diagnostic information, which are necessary for diagnoses and represent diagnostic opinions, results of medical interviews, and the like, having been obtained for disease diagnoses, are registered in accordance with clinical charts concerning diagnoses having been made in the past, and the like. FIG. 3 is an explanatory view showing an example of how diagnostic information is symbolized. By way of example, as illustrated in FIG. 3, as for the disease information, the disease names are represented by English capital letters (hereinafter referred to as the capital letters). Also, the plurality of pieces of the diagnostic information corresponding to the disease names are registered. Examples of the plurality of pieces of the diagnostic information include the diagnostic opinions, which are ordinarily obtained for the diseases, the image diagnosis opinions, which are obtained from the diagnoses having been made on the medical images represented by the medical image signals 300 obtained from various kinds of the modalities, the results of the medical interviews having been made on the patients, the results of the pathological examinations, and other kinds of information. The plurality of pieces of the diagnostic information described above are symbolized and registered. More specifically, with the symbolization, each of the diagnostic opinions is represented by a # mark, a following English small letter (hereinafter referred to as the small letter) for the disease name, and a following numeral. Also, each of the image diagnosis opinions is represented by a $ mark, a following small letter, and a following numeral. Further, each of the results of the medical interviews is represented by a % mark, a following small letter, and a following numeral. Furthermore, each of the results of the pathological examinations is represented by an & mark, a following small letter, and a following numeral. Also, each of the other kinds of information is represented by an @ mark, a following small letter, and a following numeral. In this manner, the # mark, the $ mark, the % mark, the & mark, and the @ mark represent the kinds of the diagnostic information. Also, the end numerals of the diagnostic information represent the classification of the diagnostic information into large groups.

Therefore, with respect to different disease names, an identical end numeral in the diagnostic information represents that similar information (e.g., a similar diagnostic opinion or a similar result of pathological examination) is obtained. Also, fine differences in diagnostic information are discriminated by the small letters associated with the disease names.

For example, the meanings of the numerals following the # mark for the diagnostic opinions are set such that the numeral of 1 represents a swelling of a throat, and the numeral of 2 represents fever. Also, the meanings of the numerals following the $ mark for the image diagnosis opinions are set such that the numeral of 1 represents a frosted glass-like image pattern, and the numeral of 2 represents a lumpy image pattern. Further, the meanings of the numerals following the % mark for the results of the medical interviews are set such that the numeral of 1 represents limb numbness, and the numeral of 2 represents languor. Furthermore, the meanings of the numerals following the & mark for the results of the pathological examinations are set such that the numeral of 1 represents a blood sugar level higher than a standard value, and the numeral of 2 represents bloody urine.

As illustrated in FIG. 3, in accordance with the setting described above, the diagnostic information data base 11 is prepared, which specifies the correspondence relationships between the disease information, which represents the disease names (represented by the English capital letters), and the like, and the diagnostic information, which represents the diagnostic opinions (#+small letters and numerals), the image diagnosis opinions ($+small letters and numerals), the results of the medical interviews (%+small letters and numerals), the results of the pathological examinations (&+small letters and numerals), the other kinds of information (@+small letters and numerals), and the like. Further, the disease information and the diagnostic information may be stored such that which disease information and which diagnostic information correspond to which medical image among the medical images represented by the medical image signals 300 having been utilized for the image diagnoses (i.e., the medical image signals stored in the DICOM server 31, and the like).

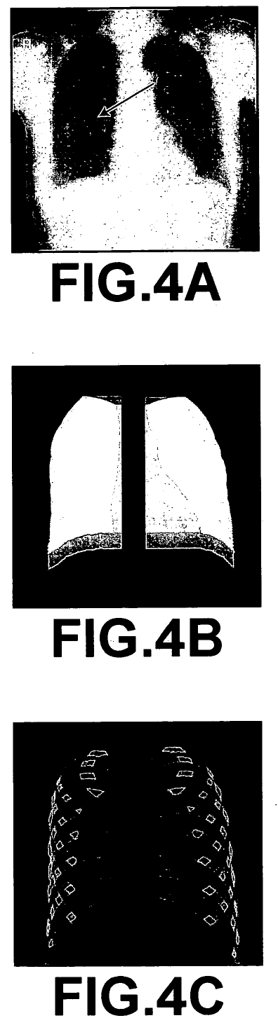

Also, together with the image diagnosis opinions, information representing the positions of tumor patterns having been found is stored. In such cases, the functions of the CAD apparatus 35 may be utilized such that, in cases where the medical doctor inputs the part indicated by the black arrow in FIG. 4A by use of a mouse device, or the like, anatomical position information of the corresponding position, or the like, is automatically symbolized and inputted. Specifically, for example, a thorax region as illustrated in FIG. 4B may be extracted from the chest image of a patient as illustrated in FIG. 4A (by use of a technique proposed by the applicant in Japanese Unexamined Patent Publication No. 2002-109550, or the like). Also, a comparison with a rib model pattern as illustrated in FIG. 4C may be made, and the information, which represents that the part indicated by the black arrow in FIG. 4A is located at the position between a seventh rib pattern and an eighth rib pattern of the right lung, may be acquired automatically. The thus acquired information may be symbolized. Alternatively, positions of abnormal patterns, which have been automatically detected by use of the functions of the CAD apparatus 35, a value representing the likelihood of a cancer, and the like, may be symbolized and stored as the image diagnosis opinions.

2) Formation of Link Information and Probability of Occurrence

How the link information is formed will be described hereinbelow. For diagnoses of predetermined diseases, approximately predetermined pieces of the diagnostic information are utilized. For example, with respect to lung diseases, the diagnoses are made in accordance with the pieces of the diagnostic information described below.
Age of the patient
Period of smoking
Abnormal results of blood examination
Abnormal results of examination of exfoliative cytology of sputum
Abnormal results of image diagnoses of simple X-ray images (or abnormal CAD results)
Abnormal results of CT examinations (or abnormal CAD results)
Presence or absence of past clinical history (tuberculosis, bronchitis, other cancers)

By way of example, ordinarily, as the period of smoking becomes long, the probability of occurrence of a cancer becomes high. In cases where opinions suspecting a lung cancer are obtained as a result of the image diagnosis of a chest X-ray image or as a result of the CAD, if the period of smoking is long, the probability of occurrence of the cancer will become high even further. Also, in cases where the opinions suspecting a lung cancer are obtained as a result of the image diagnosis of a chest X-ray image or as a result of the CAD, a CT examination, an examination of an exfoliative cytology of sputum, and the like, are performed. The correlation is thus found between each piece of the diagnostic information and the probability of occurrence of the cancer. The judgment as to the occurrence of the cancer is made from the plurality of pieces of the diagnostic information. Also, in cases where a plurality of opinions are the opinions affirming the lung cancer, the probability of occurrence of the lung cancer becomes higher. The probability of occurrence of the lung cancer is thus capable of being calculated statistically from the past diagnostic information, the cases of diagnoses as being the lung cancer, and the cases of diagnoses as not being the lung cancer.

Certain pieces of the diagnostic information, which are among the plurality of pieces of the diagnostic information for the diagnoses of lung diseases, are utilized in cases where there is a suspicion of the lung cancer and are utilized for reference in cases where the diagnoses of lung tuberculosis and bronchitis are to be made. Also, the definite judgments as to the diagnoses of the diseases as described above are often made in accordance with the plurality of the diagnostic information. It often occurs that, in cases where the probability of occurrence of a certain disease is combined with the pieces of the diagnostic information, which are associated with a high probability of occurrence, the probability of being the certain disease becomes high even further. It also often occurs that, in cases where the probability of occurrence of the certain disease is combined with the pieces of the diagnostic information, which are associated with a low probability of occurrence, the probability of being the certain disease does not alter markedly.

Therefore, the link information is formed in accordance with the past disease cases having been stored in the diagnostic information data base 11. The link information links the plurality of pieces of the diagnostic information, which are utilized at the time of diagnosis of every disease, and the disease information with each other, and/or links the plurality of pieces of the diagnostic information, which have relevancy, with one another. The link information enables the probability of occurrence of each disease to be calculated from a combination of the plurality of pieces of the diagnostic information. Also, the probability of occurrence of every disease, which is to be diagnosed, in accordance with the diagnostic information and the conditional probability of the link, which is formed among the plurality of pieces of the diagnostic information, are calculated from the past disease cases.

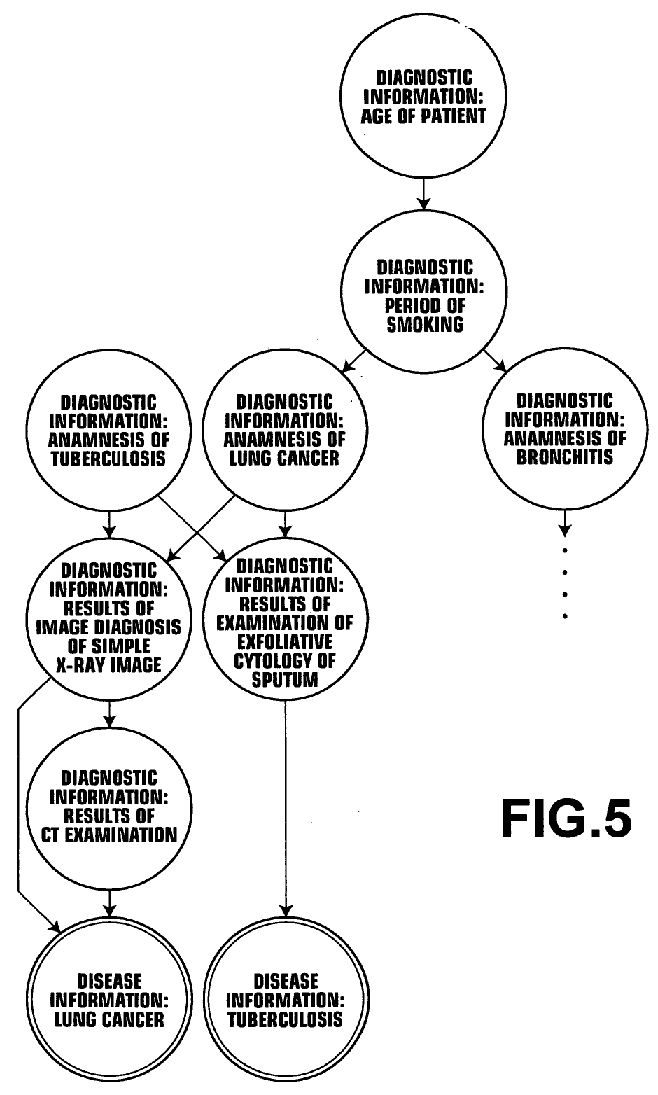

For example, the lung cancer is diagnosed synthetically in accordance with the plurality of pieces of the diagnostic information representing the period of smoking, the presence or absence of an anamnesis of the lung cancer, whether the results of the examination of the exfoliative cytology of sputum are normal or abnormal, the results of the image diagnosis of the simple X-ray image, the results of the image diagnosis of the CT image, and the like. In cases where certain pieces of the diagnostic information are utilized together for the diagnosis, the probability of occurrence may alter. Therefore, as illustrated in FIG. 5, the link is formed among the pieces of the diagnostic information, which have the relevancy. Also, certain pieces of the diagnostic information are utilized for reference in the cases of the lung cancer and in the cases of the tuberculosis, and other certain pieces of the diagnostic information are utilized in cases where a discrimination is to be made as to whether the disease is the lung cancer or is the tuberculosis. Therefore, the link information is formed such that the pieces of the diagnostic information are linked with the disease information, to which reference is made at the time of the disease diagnoses. The link information having been formed in the manner described above is stored in the link information storing means 12.

For example, in cases where the results of the examination of the exfoliative cytology of sputum are abnormal, the probability of being the lung cancer becomes high. In cases where the results of the examination of the exfoliative cytology of sputum are normal, the probability of being the lung cancer becomes low. Therefore, in accordance with the past diagnostic results having been stored in the diagnostic information data base 11, the probability of occurrence of the disease associated with the diagnostic information (examination of exfoliative cytology of sputum) and the conditional probability, which corresponds to the link of the diagnostic information (examination of exfoliative cytology of sputum) with other pieces of the diagnostic information, are calculated. The information representing the thus calculated probability of occurrence of the disease and the information representing the thus calculated conditional probability are stored in the probability storing means 13.

Also, the probability of occurrence of each disease, which probability of occurrence is obtained in cases where a synthetic judgment is made from a combination of a plurality of pieces of the diagnostic information, and the conditional probability corresponding to each link are capable of being calculated by use of the Bayes theory, or the like. The Bayes theory may be represented by the formula shown below.

$$Pr \{ B \vert A \} = \frac{Pr \{ A ⋂ B \}}{Pr \{ A \}} = \frac{Pr \{ B \} \times Pr \{ A \vert B \}}{Pr \{ B \} \times Pr \{ A \vert B \} + Pr \{ B \} \times Pr \{ A \vert B \}}$$

wherein Pr{A} represents the probability of an event A, and Pr{B} represents the probability of an event B.

Also, ordinarily, in cases where the event B is divided into r number of exclusive events, and in cases where the probabilities of the exclusive events are represented by Pr{B₁}, . . . , Pr{Bᵢ}, . . . , Pr{Bᵣ}, the probability Pr{Bᵢ|A} that the cause for the observed event A will be Bᵢ may be represented by the formula shown below.

$$Pr \text{\{} {B}_{i} \vert A \} = \frac{Pr \{ A ⋂ {B}_{i} \}}{Pr \{ A \}} = \frac{Pr \{ {B}_{i} \} \times Pr \{ A \vert {B}_{i} \}}{{\sum }_{j = 1}^{r} Pr \{ {B}_{j} \} \times Pr \{ A \vert {B}_{j} \}}$$

The probability of occurrence of each disease is calculated by use of the Bayes theory, which may be represented by the formula shown above, and in accordance with the link information as illustrated in FIG. 5. Also, it is necessary for the conditional probability to be set such that the thus obtained probability of occurrence coincides with the probability of occurrence of the disease having been obtained from the past diagnostic information.

The formation of the link information and the calculations of the probabilities (the probability of occurrence and the conditional probability) are capable of being performed in accordance with the diagnostic information having been stored in the diagnostic information data base 11 and by use of a data mining tool, or the like. For example, with respect to the relationship among the plurality of pieces of the diagnostic information or the relationship between the diagnostic information and the diseases, a decision tree may be formed automatically by use of the data mining tool, such as WAKE (Waikato Environment for Knowledge Analysis), and may be taken as the link information. Further, the probability of occurrence of a predetermined disease and the conditional probability, which are obtained from the diagnostic information, may be acquired by use of the data mining tool.

3) Diagnosis of Patient

How a patient is diagnosed by use of the link information described above will hereinbelow be described in detail.

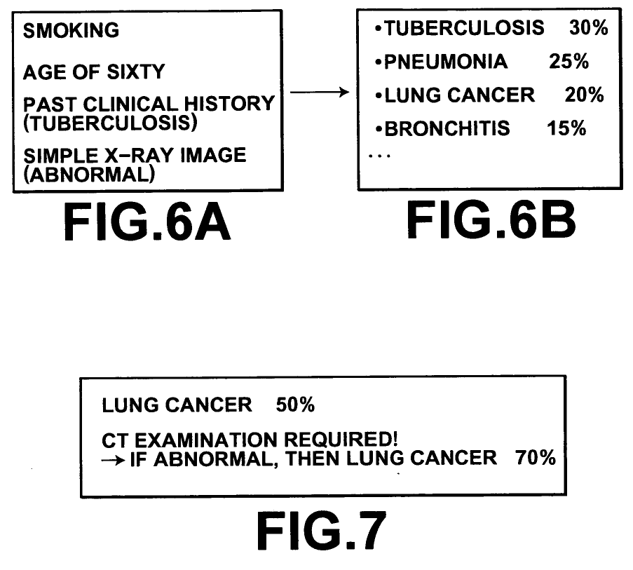

The medical doctor makes a medical examination on a new patient and inputs the diagnostic information from the input means 14. For example, as illustrated in FIG. 6A, in the cases of the diagnosis of the lung diseases, the medical doctor inputs the pieces of the diagnostic information representing the period of smoking, the age (sixty), the past clinical history (tuberculosis), and the results of the image diagnosis of the simple X-ray image. Also, by use of the inputted pieces of the diagnostic information and the link information, the probability acquiring means 15 calculates all of the probabilities of occurrence of the diseases which are diagnosed from the diagnostic information. The probabilities of occurrence of the diseases are calculated by use of the Bayes theory described above and in accordance with the link information. Specifically, software functions of the Bayesian net, and the like, may be utilized.

The calculations of the probabilities of occurrence of the diseases are made with respect to all pieces of the disease information linked with the inputted diagnostic information. As illustrated in FIG. 6B, the disease information may be displayed on the displaying means 16 in the order of decreasing probability of occurrence of each disease.

Also, the diagnosis aiding apparatus 1 may investigate the diagnostic information, which is linked with the disease information of each disease having been retrieved as a candidate, by following the link information. Further, the diagnosis aiding apparatus 1 may automatically search for a certain piece of the diagnostic information, which certain piece is contained in the diagnostic information linked with the disease information and which has not been manually inputted, from the own computer and from computers of the systems located in the hospital and may automatically input the thus searched piece of the diagnostic information from the input means 14.

For example, the computers expected to store the diagnostic information, such as the DICOM server 31, the storage server 21 provided with the electronic clinical charts 200, the RIS terminal 32 located at each consultation department, and the HIS terminal 22 located at each consultation department, are registered previously in the diagnosis aiding apparatus 1. In cases where the necessary pieces of the diagnostic information are not found in the own computer, the diagnosis aiding apparatus 1 successively searches for the necessary pieces of the diagnostic information through the network 36 and from the registered computers (such as the DICOM server 31, the storage server 21, the RIS terminal 32, and the HIS terminal 22).

Specifically, for example, as the link information at the time of the diagnosis of the lung cancer, the plurality of pieces of the diagnostic information representing the results of the image diagnosis of the simple X-ray image, the results of the image diagnosis of the CT image, whether the results of the examination of the exfoliative cytology of sputum are normal or abnormal, the presence or absence of an anamnesis of the lung cancer, and the age of the patient may be linked. Also, the medical doctor may manually input the piece of the diagnostic information, which represents that the results of the blood examination are abnormal, and the piece of the diagnostic information, which represents that the age of the patient is sixty, and may not input the other pieces of the diagnostic information. In such cases, the diagnosis aiding apparatus 1 automatically searches for the other pieces of the diagnostic information (representing the results of the image diagnosis of the simple X-ray image, the results of the image diagnosis of the CT image, whether the results of the examination of the exfoliative cytology of sputum are normal or abnormal, and the presence or absence of an anamnesis of the lung cancer) from the own computer. Also, the diagnosis aiding apparatus 1 automatically searches for the other pieces of the diagnostic information through the network 36 and from the computers located in the hospital. In cases where the piece of the diagnostic information representing the results of the image diagnosis of the CT image is not found from the DICOM server 31 and the other computers as a result of the searching for the piece of the diagnostic information representing the results of the image diagnosis of the CT image, the piece of the diagnostic information representing the results of the image diagnosis of the CT image is kept uninputted. In cases where the piece of the diagnostic information, which represents that the results of the examination of the exfoliative cytology of sputum are abnormal, is found from the electronic clinical charts 200, which have been stored in the storage server 21, as a result of the searching for the piece of the diagnostic information representing whether the results of the examination of the exfoliative cytology of sputum are normal or abnormal, the piece of the diagnostic information, which represents that the results of the examination of the exfoliative cytology of sputum are abnormal, is automatically inputted from the input means 14.

When the searching for all of the pieces of the diagnostic information having been linked as the link information has been finished, the probability acquiring means 15 calculates the probability of occurrence of the lung cancer in accordance with the inputted pieces of the diagnostic information.

Also, the selection means 17 extracts the pieces of the diagnostic information, which have not been found from the own computer and from the other computers through the network 36 and are kept uninputted, from the diagnostic information having been linked as the link information for the cancer. Further, the probability acquiring means 15 calculates the probability of occurrence of the cancer, which is obtained in cases where one of the uninputted pieces of the diagnostic information is added to the inputted pieces of the diagnostic information. The selection means 17 thus selects certain pieces of the diagnostic information from the uninputted pieces of the diagnostic information, each of the certain pieces of the diagnostic information being of the kind such that, in cases where the certain piece of the diagnostic information is added to the inputted pieces of the diagnostic information and utilized for the acquisition of the probability of occurrence, the probability of occurrence higher than the probability of occurrence, which has been acquired before the certain piece of the diagnostic information is added, is obtained. The certain pieces of the diagnostic information having thus been selected are sorted in the order of the decreasing probability of occurrence. Also, the display device 18 displays an instruction for requesting the inputting of the certain piece of the diagnostic information, which has been sorted as the first certain piece of the diagnostic information.

For example, the probability of occurrence of the lung cancer, which has been acquired by the probability acquiring means 15 in accordance with the pieces of the diagnostic information having been inputted by the medical doctor and the pieces of the diagnostic information having been automatically searched, may be 50%. Also, the certain piece of the diagnostic information, which has been selected by the selection means 17 from among the pieces of the diagnostic information having been linked as the link information for the lung cancer and is associated with the highest probability of occurrence of the lung cancer, may be the CT examination. In such cases, as illustrated in FIG. 7, the instruction requesting the examination with the CT scanner is displayed.

In the embodiment described above, among the pieces of the diagnostic information having been linked with the disease information for the lung cancer, the pieces of the diagnostic information, which have been inputted manually, and the pieces of the diagnostic information, which have been found as a result of the searching from the computers, are utilized for the calculation of the probability of occurrence. Alternatively, firstly, the probability of occurrence may be calculated by the probability acquiring means 15 by use of only the pieces of the diagnostic information, which have been inputted manually by the medical doctor. Thereafter, the uninputted pieces of the diagnostic information may be selected by the selection means 17 from the pieces of the diagnostic information having been linked as the link information. Further, the uninputted pieces of the diagnostic information having thus been selected may be searched from the own computer and the computers of the information systems located in the hospital.

Also, for example, in cases where the searched piece of the diagnostic information is the one representing the results of the CT examination, and in cases where the CT image of the corresponding patient has been found by the diagnosis aiding apparatus 1 from the DICOM server 31, the image signal representing the CT image may be sent into the CAD apparatus 35, and image analysis processing may be performed by the CAD apparatus 35 on the CT image. Further, the results of the image analysis may be symbolized and inputted from the input means 14 as the diagnostic information. Alternatively, the image signal representing the CT image of the corresponding patient may be sent into the diagnosis aiding apparatus 1, and the results of the image diagnosis, which has been made by the medical doctor on the CT image having been displayed on a display screen of the diagnosis aiding apparatus 1, may be inputted from the input means 14 as the diagnostic information.

As described above in detail, with the embodiments of the diagnosis aiding apparatus in accordance with the present invention, the computer actively searches the pieces of the information, which are to be utilized additionally, and the accuracy with which the diagnosis is made is capable of being enhanced. Also, the load to the medical doctor is capable of being kept light.

Further, with the embodiments of the diagnosis aiding apparatus in accordance with the present invention, the probabilities of occurrence of all of the diseases, which have possibility, are calculated from the inputted diagnostic information. Therefore, the diseases other than the diseases expected by the medical doctor are capable of being recognized as the candidates.

Furthermore, with the embodiments of the diagnosis aiding apparatus in accordance with the present invention, the pieces of the diagnostic information, which are to be utilized additionally, are capable of being displayed, and a guide of an examination, or the like, is capable of being instructed to the medical doctor.

What is claimed is:

1. A diagnosis aiding apparatus, comprising:
i) link information storing means for storing a plurality of pieces of diagnostic information, which are necessary for disease diagnoses, and at least one piece of disease information, which represents a disease to be diagnosed in accordance with the diagnostic information, and storing link information with respect to each piece of the disease information,
the link information linking the plurality of pieces of the diagnostic information with one another in accordance with relationships among the plurality of pieces of the diagnostic information, which are to be utilized at the time of a diagnosis of each disease, and/or linking the plurality of pieces of the diagnostic information and the disease information with each other in accordance with relationships between the plurality of pieces of the diagnostic information and the disease information,
ii) probability storing means for previously storing a probability of occurrence, with which the disease occurs in accordance with the plurality of pieces of the diagnostic information, and a probability of occurrence, with which the disease occurs in accordance with a combination of the plurality of pieces of the diagnostic information corresponding to the link information,
iii) input means for inputting at least one piece of the diagnostic information among the plurality of pieces of the diagnostic information, and
iv) probability acquiring means for retrieving the probability of occurrence, which is associated with the diagnostic information having been inputted from the input means, from the probability storing means, and acquiring the probability of occurrence with respect to each disease by use of the thus retrieved probability of occurrence and in accordance with the link information of each piece of the disease information.

2. An apparatus as defined in claim 1 wherein the apparatus further comprises displaying means for displaying the disease information in the order of decreasing probability of occurrence of each disease, which probability of occurrence has been acquired by the probability acquiring means.

3. An apparatus as defined in claim 1 wherein the apparatus further comprises selection means for selecting a certain piece of the diagnostic information, which is other than the at least one piece of the diagnostic information having been inputted from the input means, from among the plurality of pieces of the diagnostic information, which are linked by the link information and are to be used at the time of the diagnosis of a predetermined disease,
the certain piece of the diagnostic information being of the kind such that, in cases where the certain piece of the diagnostic information is added to the at least one piece of the diagnostic information having been inputted from the input means and is utilized for the acquisition of the probability of occurrence of the predetermined disease made by the probability acquiring means, the probability of occurrence of the predetermined disease, which probability is higher than the probability of occurrence of the predetermined disease having been acquired from the probability acquiring means by use of the at least one piece of the diagnostic information having been inputted from the input means, is acquired from the probability acquiring means.

4. An apparatus as defined in claim 2 wherein the apparatus further comprises selection means for selecting a certain piece of the diagnostic information, which is other than the at least one piece of the diagnostic information having been inputted from the input means, from among the plurality of pieces of the diagnostic information, which are linked by the link information and are to be used at the time of the diagnosis of a predetermined disease,
the certain piece of the diagnostic information being of the kind such that, in cases where the certain piece of the diagnostic information is added to the at least one piece of the diagnostic information having been inputted from the input means and is utilized for the acquisition of the probability of occurrence of the predetermined disease made by the probability acquiring means, the probability of occurrence of the predetermined disease, which probability is higher than the probability of occurrence of the predetermined disease having been acquired from the probability acquiring means by use of the at least one piece of the diagnostic information having been inputted from the input means, is acquired from the probability acquiring means.

5. A diagnosis aiding method, comprising the steps of:
i) storing a plurality of pieces of diagnostic information, which are necessary for disease diagnoses, and at least one piece of disease information, which represents a disease to be diagnosed in accordance with the diagnostic information,
ii) storing link information with respect to eachpiece of the disease information,
the link information linking the plurality of pieces of the diagnostic information with one another in accordance with relationships among the plurality of pieces of the diagnostic information, which are to be utilized at the time of a diagnosis of each disease, and/or linking the plurality of pieces of the diagnostic information and the disease information with each other in accordance with relationships between the plurality of pieces of the diagnostic information and the disease information,
iii) previously storing a probability of occurrence, with which the disease occurs in accordance with the plurality of pieces of the diagnostic information, and a probability of occurrence, with which the disease occurs in accordance with a combination of the plurality of pieces of the diagnostic information corresponding to the link information,
iv) inputting at least one piece of the diagnostic information among the plurality of pieces of the diagnostic information,
v) retrieving the probability of occurrence, which is associated with the diagnostic information having been inputted from the input means, from the probability storing means, and
vi) acquiring the probability of occurrence with respect to each disease by use of the thus retrieved probability of occurrence and in accordance with the link information of each piece of the disease information.

6. A computer program for operating a computer provided with:
i) link information storing means for storing a plurality of pieces of diagnostic information, which are necessary for disease diagnoses, and at least one piece of disease information, which represents a disease to be diagnosed in accordance with the diagnostic information, and storing link information with respect to each piece of the disease information,
the link information linking the plurality of pieces of the diagnostic information with one another in accordance with relationships among the plurality of pieces of the diagnostic information, which are to be utilized at the time of a diagnosis of each disease, and/or linking the plurality of pieces of the diagnostic information and the disease information with each other in accordance with relationships between the plurality of pieces of the diagnostic information and the disease information, and
ii) probability storing means for previously storing a probability of occurrence, with which the disease occurs in accordance with the plurality of pieces of the diagnostic information, and a probability of occurrence, with which the disease occurs in accordance with a combination of the plurality of pieces of the diagnostic information corresponding to the link information,
wherein the computer program causes the computer to function as:
a) input means for inputting at least one piece of the diagnostic information among the plurality of pieces of the diagnostic information, and
b) probability acquiring means for retrieving the probability of occurrence, which is associated with the diagnostic information having been inputted from the input means, from the probability storing means, and acquiring the probability of occurrence with respect to each disease by use of the thus retrieved probability of occurrence and in accordance with the link information of each piece of the disease information.
